SEQUENCE LISTING (5 sequences)

SEQ ID NO: 1 - protein, 11 aa, Artificial Sequence
  Description of Artificial Sequence; note = synthetic construct
CGGLGPAGGLC

SEQ ID NO: 2 - protein, 6 aa, Artificial Sequence
  Description of Artificial Sequence; note = synthetic construct
CRGDSP

SEQ ID NO: 3 - protein, 7 aa, Artificial Sequence
  Description of Artificial Sequence; note = synthetic construct
CRGDSPC

SEQ ID NO: 4 - protein, 11 aa, Artificial Sequence
  Description of Artificial Sequence; note = synthetic construct
CGGLGPAGGKG

SEQ ID NO: 5 - protein, 5 aa, Artificial Sequence
  Description of Artificial Sequence; note = synthetic construct
YIGSR

(19) United States
(12) Patent Application Publication
(10) Pub. No.: US 2007/0167354 A1
(43) Pub. Date: Jul. 19, 2007

(54) Hydrogels for modulating cell migration and matrix deposition

(76) Inventors: Chad E. Kennedy, Chandler, AZ (US); Gholam Reza Ehteshami, Scottsdale, AZ (US); Doris Hom, Mesa, AZ (US); Stephen Massia, Phoenix, AZ (US)

Correspondence Address: NEEDLE & ROSENBERG, P.C., SUITE 1000, 999 PEACHTREE STREET, ATLANTA, GA, 30309-3915, US

(21) Appl. No.: 10/569,905

(22) PCT Filed: Aug. 27, 2004
(86) PCT No.: PCT/US04/28000

§ 371 Date: Jan. 3, 2007

Related U.S. Application Data
(60) Provisional application No. 60/499,634, filed on Aug. 28, 2003.

(51) Int. Cl. A61K 38/14; A61K 38/10; A61K 38/08; C07K 9/00

(52) U.S. Cl. 514/8; 530/322; 514/14; 514/17

(57) ABSTRACT

A biodegradable matrix for inducing cell migration therein, wherein two peptides are covalently linked to the matrix, a first peptide being cleavable by natural proteases and the other comprising a cell-attracting peptide. The first peptide is cleavable by tissue metalloproteinases. The second peptide contains the cell-attracting peptide RGD. Also disclosed is an implant containing the biodegradable matrix.

### TECHNICAL FIELD

The invention relates to biocompatible, biodegradable hydrogels cross-linked with two peptide signals for cell migration and matrix deposition, and more specifically to dextran with cross-links of an RGD peptide and/or an enzyme cleavable peptide.

### BACKGROUND

Hydrogels have been gaining significance as an effective biomaterial; their interactions with biological tissues or functions demonstrate high biocompatibility. Specifically, a hydrogel is a hydrophilic polymer network that can store a percentage of water. This is created by forming cross links between polymeric strands, which can be chemically modified to react using light, heat, or pH changes. Dextran hydrogels can be prepared in this manner.

Of the many biocompatible gels, dextran gels have been recognized as a good material for experimental use and have been FDA approved. As a natural polymer, dextran is a polysaccharide that is biodegradable through hydrolysis or enzymatic reactions. Additionally, its hydroxyl functional groups allow for chemical modification to either form cross-links in hydrogel formation or to attach molecules. As a material, dextran is inexpensive and easy to manipulate.

Degradation of hydrogels depends on hydrolysis of either the cross-links or the polymer backbone. This process is unique for the different hydrogel compositions. Some may be synthesized with targeted degradation of the cross-links or the backbone. Usual degradation of polymers relies on adsorption of medium on the polymer surface, diffusion of medium into the polymer, chemical reactions, diffusion of degradation products, and description of degradation products. For the most part, degradation is carried out by specific enzymatic hydrolysis of hydrogel components.

Endo-dextranase has been cross-linked in dextran methacrylated hydrogels in order to degrade the polymer. The degradation rate depended on the cross-link density and the amount of dextranase present. Lower cross-link density correlated with shorter degradation times. A similar method was employed to produce degradation-controlled release of proteins. Enzymatically-degrading dextran hydrogels were made by co-entrapment of dextranase. In addition, chemically degrading gels of hydroxyethyl methacrylated dextran have been prepared to compare degradation time with cross-linking.

Cell migration occurs in response to extracellular signals and phenotypic preference; this concept is an established notion in cell biology. Through chemotaxis, the cells move in a direction controlled by a gradient of diffusible chemicals. In cases of injury, this effect is brought about by signaling from the immune system. For vascular injury following angioplasty, the response of smooth muscle cells to injury signals leads to accumulation of neointimal smooth muscle cells. Cell migration studies are typically conducted in experimental assays with modified Boyden chambers. These chambers have inserts that separate cells from chemo attractants in the lower well. The chemical signaling causes the migration of cells through micron size pores; these pore sizes and density may be specific for each type of cell.

The movement of cells to the extracellular matrix is facilitated by the release of matrix metalloproteinase (MMPs) to dissolve numerous extracellular substrates. These extracellular proteinases are able to degrade structural proteins of the extracellular matrix (ECM), cleave cell surface molecules, and other proteins. This regulates cell behavior in response to physiological processes including embryonic development, wound repair, cancer, and tissue morphogenesis. Since their identification, several classes of MMPs have been discovered to function in different ways; some specifically target amino acid residues to remove barriers to invasion.

What is needed is an improved biodegradable matrix that will encourage in-growth of tissue.

### SUMMARY OF THE INVENTION

It is an object of this invention to provide a biocompatible, biodegradable scaffold for tissue ingrowth. In one embodiment, there is a biodegradable matrix for inducing cell migration therein, wherein two peptides are covalently linked to the matrix, a first peptide being cleavable by natural proteases and the other comprising a cell-attracting peptide. The matrix can be dextran. The dextran matrix can be glycidyl methacrylate dextran. In one embodiment, the dextran has a molecular weight of 40 kDa. The first peptide can be comprised of at least the sequence CGGLGPAGGLC (SEQ ID NO:1). The second peptide comprises in part the sequence RGD. The second peptide can include CRGDSP (SEQ ID NO: 2). The second peptide can include CRGDSPC (SEQ ID NO: 3).

In another embodiment, there is a method of preparing a dextran hydrogel suitable for cellular in-growth. The method has the steps of providing a dextran; combining the dextran with a cleavable peptide and a peptide capable of attracting cells to produce a conjugated dextran; combining the conjugated dextran with acryloylated dextran; adding to the dextran mixture a polymerization initiator; and activating the initiator to form a hydrogel. The dextran can be dextran 40. The cleavable peptide can include CGGLGPAGGLC (SEQ ID NO: 1). The peptide capable of attracting cells comprises in part RGD. The RGD peptide further comprises CRGDSP (SEQ ID NO: 2). Alternatively, the RGD peptide can comprise CRGDSPC (SEQ ID NO: 3). The conjugated peptide can be in higher proportion than the acryloylated dextran.

In yet another embodiment, there is a method of preparing a hydrogel suitable for promoting cellular in-growth. This method has the steps of providing dextran and combining the dextran with dimethylsulfoxide (DMSO), dimethylaminopyridine (DMAP) and glycol methacrylate (GMA) to form glycidyl methacrylate dextran. The glycidyl methacrylate dextran is next combined with acryloylated dextran, this dextran mixture is combined with a polymerization initiator and with at least two peptides, a first peptide capable of attracting cells and a second peptide being degradable by cellular proteases, in a dilute electrolyte solution; and finally applying energy to polymerize the mixture, thus producing a hydrogel. DMSO, DMAP and GMA can be added sequentially to the dextran, which combination can be followed by mixing at room temperature until the solution is completely dissolved. This mixing step can be followed by adding hydrochloric acid to neutralize the solution and stop the reaction. After mixing in the acryloylated dextran, the combination can be dialyzed. In some embodiments, the content of conjugated dextran is greater than the content of acryloylated dextran.

In still another embodiment, there is provided an implant including a dextran hydrogel, wherein two peptides are covalently linked to the dextran, a first peptide being cleavable by natural proteases and the other comprising a cell-attracting peptide. The dextran can have a molecular weight of 40 kDA. The dextran can be glycidyl methacrylate dextran. The first peptide has at least the sequence CGGLGPAGGLC (SEQ ID NO: 1). The second peptide comprises at least the sequence RGD. The RGD sequence further comprises CRGDSP (SEQ ID NO: 2). The RGD sequence can further include CRGDSPC (SEQ ID NO: 3).

In yet another embodiment, there is a dextran matrix for inducing cell migration therein, wherein a peptide is covalently linked to the matrix and the peptide is cleavable by natural proteases. The cleavable peptide can be CGGLGPAGGLC (SEQ ID NO: 1), CGGLGPAGGKG (SEQ ID NO: 4), or a combination thereof. The dextran can be glycidyl methacrylated dextran. The dextran can have a molecular weight of 40 dKa.

In yet another embodiment, there is a dextran matrix for inducing cell migration therein, wherein a peptide is covalently linked to the matrix and attracts cells. The dextran can be glycidyl methacrylated dextran. The dextran can have a molecular weight of 40 dKa. The peptide can be CRGDSP (SEQ ID NO: 2), CRGDSPC (SEQ ID NO: 3), or a combination thereof.

### BRIEF DESCRIPTION OF THE DRAWINGS

FIG. 1 is a bar graph showing the percent of image area covered by cells allowed to migrate for 4, 24 and 48 hours. Different bars represent the no-gel control, 20% acryloylated dextran(a-dex)/80% conjugated dextran(c-dex), 40% a-dex/60% c-dex, 60% a-dex/40% c-dex and 80% a-dex/20% c-dex. The conjugated dextran was conjugated with the protease cleavable peptide CGGLGPAGGKG (SEQ ID NO: 4).

FIG. 2 is a bar graph showing bovine endothelial cells (BEC) adhesion to hydrogels as a function of covalently linked CRGDSPC (SEQ ID NO: 3) peptide concentration. Error bars represent means (+,−) and standard deviations for n=3.

FIG. 3 is a bar graph showing BEC adhesion to hydrogels as a function of covalently linked CRGDSPC (SEQ ID NO: 3) peptide concentration. Cell numbers were normalized to the initial seeding density of the control surfaces. Error bars represent means and standard deviations for n=3.

FIGS. 4a-4e are digitized images of BEC 24 hr after seeding on CRGDSPC (SEQ ID NO: 3) peptide hydrogel, including the following formulations: (a) 0% peptide cross-linked dextran hydrogel, (b) 10% peptide-cross-linked dextran hydrogel, (c) 30% peptide cross-linked hydrogel, (d) cell culture plastic dishes (control), and (e) dextran-coated cell culture plastic plates.

FIGS. 5a-5e are digitized images of BEC 24 hr after seeding on CRGDSPC (SEQ ID NO: 3) peptide with a) 0% peptide in cross-linked dextran hydrogel, (b) 10% peptide in cross-linked dextran hydrogel, (c) cell culture plastic dishes (control), and (d) dextran-coated cell culture plastic plates.

FIGS. 6a-6c are digitized images of BEC 24 hr after seeding on CRGDSPC (SEQ ID NO: 3) peptide (a) 0% peptide cross-linked dextran hydrogel, (b) 50% peptide cross-linked dextran hydrogel and (c) cell culture plastic dishes (control).

### DETAILED DESCRIPTION

This application features a new kind of biodegradable hydrogel matrix. This dextran hyrdrogel is adapted for controlled growth of tissue by adding a proteolytically cleavable peptide and and/or RGD-containing peptide. The introductions of the cleavable peptide (by MMP) and an RGD-containing peptide are intended to modulate the migration of cells. The dextran hydrogel was synthesized in a combination of steps and tested. Cell migration assays were used to validate the concept. More new hydrogels, preferably polysaccharide based hydrogels, were synthesized and tested in vitro. These hydrogels were made from cross-linking acryloylated dextrin with homo mono- and bi-functional active molecules, preferably peptide, having one or two cysteines at the ends. The homo bi-functional peptides contain a MMP sensitive or cell adhesion domains forming cross links that promotes cell-induced adhesion and enzymatic degradation of the hydrogel. By changing the percent compositions of these peptide cross-links with respect to the total available cross-links, the extent of cell adhesion and migration through the gel can be regulated.

The hydrogel can also include absorbed medicaments, enhancing chemicals, proteins, and the like, for stimulating local angiogenesis, cell contractility, cell growth, in addition to cell migration, for example. These can include, for example, aFGF (acidic fibroblast growth factor), VEGF (vascular endothelial growth factors), tPA (tissue plasminogen activator), BARK (β-adrenergic receptor kinase), β-blockers, etc. Heparin, or other anticoagulants, such as polyethylene oxide, hirudin, and tissue plasminogen activator, can also be incorporated into the hydrogel prior to implantation in an amount effective to prevent or limit thrombosis.

### Definitions

“Biomaterial” as generally used herein refers to a material intended to interface with biological systems to evaluate, treat, augment, or replace any tissue, organ or function of the body depending on the material, either permanently or temporarily. The terms “biomaterial” and “matrix” are used synonymously herein and mean a cross-linked polymeric network which, depending of the nature of the matrix, can be swollen with water but not dissolved in water, i.e. form a hydrogel which stays in the body for a certain period of time fulfilling certain support functions for traumatized or defective tissue.

“Functionalize” as generally used herein refers to modifying a molecule in a manner that results in the attachment of a functional group or moiety. For example, a molecule may be Functionalize by the introduction of a molecule which makes the molecule a strong nucleophile or a conjugated unsaturation. Preferably a molecule, for example dextran, is Functionalize to become a thiol, amine, acrylate, or quinone.

“Adhesion site or cell attachment site” as generally used herein refers to a peptide sequence to which a molecule, for example, an adhesion-promoting receptor on the surface of a cell, binds. Examples of adhesion sites include, but are not limited to, the RGD sequence from fibronectin and the YIGSR (SEQ ID NO: 5) sequence from laminin. Preferably adhesion sites are incorporated into the biomaterial by including a substrate domain crosslinked to a matrix.

“Biological activity” as generally used herein refers to functional events mediated by a protein of interest. In some embodiments, this includes events assayed by measuring the interactions of a polypeptide with another polypeptide. It also includes assaying the effect which the protein of interest has on cell growth, differentiation, death, migration, adhesion, interactions with other proteins, enzymatic activity, protein phosphorylation or dephosphorylation, transcription, or translation.

“Sensitive biological molecule” as generally used herein refers to a molecule that is found in a cell, or in a body, or which can be used as a therapeutic for a cell or a body, which may react with other molecules in its presence. Examples of sensitive biological molecules include, but are not limited to, peptides, proteins, nucleic acids, and drugs. Biomaterials can be made in the presence of sensitive biological materials, without adversely affecting the sensitive biological materials.

“Cross-linking” as generally used herein means the formation of covalent linkages. However, it may also refer to the formation of non-covalent linkages, such as ionic bonds, or combinations of covalent and non-covalent linkages.

“Polymeric network” as generally used herein means the product of a process in which substantially all of the monomers, oligo- or polymers are bound by intermolecular covalent linkages through their available functional groups to result in one huge molecule.

“Physiological” as generally used herein means conditions as they can be found in living vertebrates. In particular, physiological conditions refer to the conditions in the human body such as temperature, pH, etc. Physiological temperatures means in particular a temperature range of between 35° C. to 42° C., preferably around 370° C.

“Swelling” as generally used herein refers to the increase in volume and mass by uptake of water by the biomaterial. The terms “water-uptake” and “swelling” are used synonymously throughout this application.

“Equilibrium state” as generally used herein as the state in which a hydrogel undergoes no mass increase or loss when stored under constant conditions in water.

“Dextran” as used herein refers to polymers of α-D-glucopyranosyl units, differing only in degree of branching and chain length. It is contemplated that this invention can utilize molecular weights kDa from 10,000 to 1,000,000 kilo Daltons (kDa). As such, the dextran may have molecular weights of 20, 40, 70, 100 or other. This invention is illustrated with dextran 40 (40 kDa). The dextran used herein should be oxidized or otherwise functionalized to accept cross links. The dextran used herein is 20% oxidized. Dextrans oxidized at least 5% and as much as 50% may be used. Obviously that includes dextrans with e.g., 25%, 30%, and 40% of their monomers oxidized.

One peptide of the present invention is a cleavable peptide and is specifically designed as a substrate for natural proteases. Preferably the sequences in the peptide are substrates for enzymes that are involved in cell migration (e.g., as substrates for enzymes such as collagenase, plasmin, elastase and particularly metalloproteinases), although suitable domains are not limited to these sequences. Two particularly preferred sequences are SEQ ID NO: 1 and SEQ ID NO 4: The degradation characteristics of the gels can be manipulated by changing the composition of the peptides that serve as the cross-linkers. For instance, the cleavable peptide may be one that is degradable by one enzyme and not another. Also the sequence of the cleavable peptide can be varied to change the Kₘ or k_{cat}, or both, of the enzymatic reaction.

The biodegradable and biocompatible polymer is selected from the group consisting of poly(lactide)s, poly(glycolide)s, poly(lactide-co-glycolide)s polyanhydrides, polyorthoesters, polyetheresters, polycaprolactone, polyesteramides, blends and copolymers thereof.

The polymeric matrix of this invention can include one or more other synthetic or natural polymers. Suitable polymers include those that are compatible with the cells or genetic material. They can be biostable or biodegradable. These include, but are not limited to, fibrins, collagens, alginates, polyacrylic acids, polylactic acids, polyglycolic acids, celluloses, hyaluronic acids, polyurethanes, silicones, polycarbonates, and a wide variety of others typically disclosed as being useful in implantable medical devices. Preferably, the polymers are hydrophilic.

The polymers of this invention include most preferably dextran.

### EXAMPLE 1

### Synthesis of Peptides

Four peptides including CGGLGPAGGKC (cleavable peptide)(SEQ ID NO: 4), CRGDSPC (homo bi-functional RGD peptide)(SEQ ID NO: 3), CGGLGPAGGLC (cleavable peptide)(SEQ ID NO: 1) and CRGDSP (mono functional RGD peptide)(SEQ ID NO: 2) were synthesized by the ASU Protein Chemistry Facility. These polypeptides were synthesized on Fmoc-XAL-PEG-polystyrene resin on a Miligen-Biosearch 9050 peptide synthesizer using standard Fmoc chemistry with HATU (N-[(dimethylamino)-1H-1,2,3-triazolo[4,5-b]pyridino-1-ylmethylene]-N-methylmethanaminium hexafluorophosphate N-oxide, Applied Biosystems Foster City, CA) as the activator. After synthesis, peptides were cleaved from the synthesis resin with trifluoroacetic acid containing 2.5% triisopropyl silane and 2.5% ethanedithiol as scavengers. The peptides were then precipitated and washed 3 times with diethyl ether and vacuum dried overnight. The molecular masses of the peptides were confirmed using mass spectroscopy.

### EXAMPLE 2

### Synthesis of Conjugated Dextran and Dextran Hydrogel

Functionalized dextran (20% oxidized) was reacted with peptide (SEQ ID NO: 4) in phosphate buffer. For a yield of 100 mg of conjugated dextran, 100 mg peptide and 100 mg dextran were used. The two reactants were mixed well in 2 mL of phosphate buffer. The reaction proceeded for 40 hours. Six hours prior to completion, 0.02 g NaBH₄ was added to reduce possible disulfide bond formation between the peptides. To purify the product, the entire solution was placed in a dialysis bag (SnakeSkin Pleated Dialysis Tubing Product, 10,000 MW cut-off, Pierce Biotechnology, Rockford, Ill.). Dialysis ran for two days, with the water changed every few hours to facilitate the diffusion of unreacted elements.

The conjugated dextran (c-dex) was reacted with acryloylated dextran (a-dex) (degree of substitution of 20%) in phosphate buffered saline (Gibco, without calcium chloride or magnesium chloride). The mixtures had the following dextran compositions: 20% a-dex/80% c-dex, 40% a-dex/60% c-dex, 60 a-dex/40% c-dext and 80% a-dex-/20% c-dex. A photoinitiator was prepared through dissolution of 300 mg 2,2,dimethoxy-2-phenyl-acetophenone (Aldrich 19,611-8) in 1 mL of N-vinylpyrrolidone (Aldrich V340-9. An ultraviolet ray lamp (G-100AP Ultra-Violet Products, Inc., Upland, Calif.) of peak wavelength 370 nm was used to initiate cross-linking between the conjugated dextran and acryloylated dextran. Cross-linking of the conjugated dextran and acryloylated dextran produced white, opaque gels after exposure to UV for a few minutes. Without addition of the photoinitiator, the pre-gel mixture remained a viscous liquid.

### EXAMPLE 3

### Cell Migration Assay

Bovine endothelial cells (CRL GMC 7372B, NIGMS Human Genetic Cell Repository Camden, N.J.) were maintained in minimum essential medium (MEM; Gibco, Invitrogen, Carlsbad, Calif. 10370-021) supplemented with 10% FBS (Gemini-Bioproducts, 100-106), 1% antibiotic-antimycotic (Gibco, 152-062), and 1% L-glutamine (Gibco, 25030-81) in 5% CO₂.

The cell migration assay was conducted on a 24-well plate (BD Falcon HTS FluoroBlok™ Multiwell Insert System, 8 μm pore size) with a light-opaque membrane of PET. The plates were first pre-wetted with 0.5 mL of 1% Triton X-100 in PBS to improve the affinity of the polystyrene sides of the insert to the hydrogel. The dextran and photoiniator were dissolved in PBS at 20 weight %. To produce a 1 mm thick gel, 30 μL of the gel mixture was pipetted into each well.

Preparation of the cells for use involved exposing cells to a 3 mL solution containing fluorescent probe (CellTracker™ probes by Molecular Probes, Eugene, OR C-2925, C-7025). The cells were incubated for 30 min, the working solution was replaced with fresh media afterwards, and incubation continued for 30 min. Cells were detached using 0.25% trypsin, 1% EDTA, then pelleted, counted and resuspended in serum-free medium at a concentration of 1×10⁶ cell/mL. The cells were seeded on top of the hydrogels in a volume of 300 μL. The lower wells were filled with 1000 μL of supplemented medium via the access ports.

Using IMAQ Vision Builder 6 (National Instruments, Austin, Tex.), the percentage of migrated cells was determined as the average of three images. The cell area was represented as the percentage of the image area. ANOVA techniques were performed using an α=0.05 to determine significant differences between hydrogel ratios.

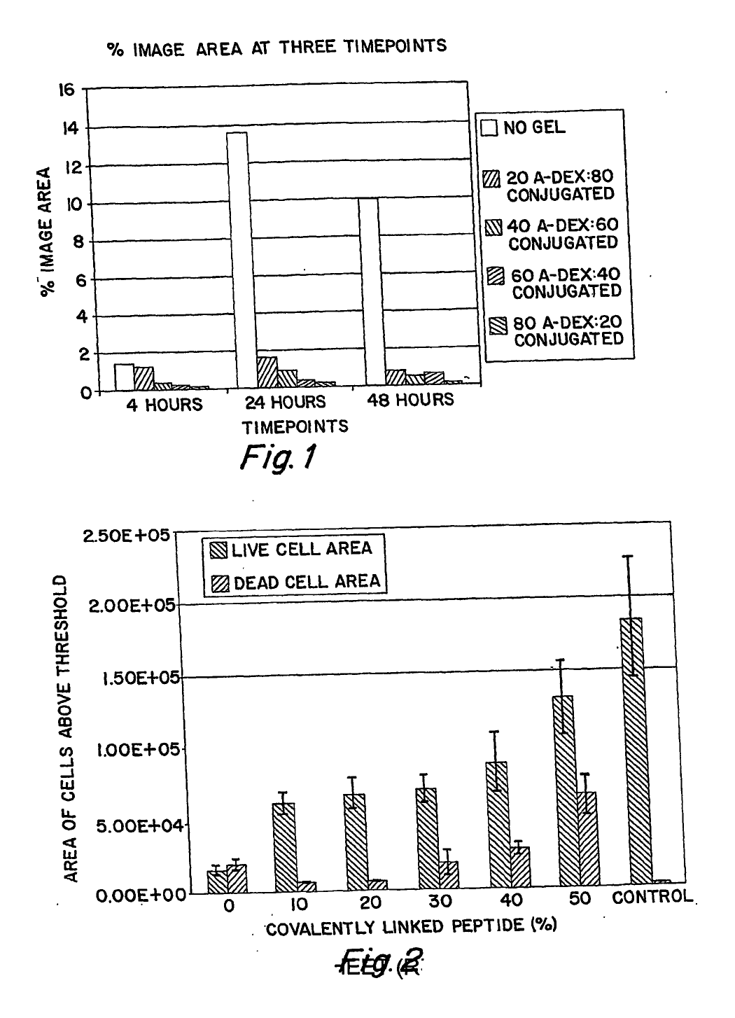

FIG. 1 shows the percentage of cells covering the image area observed at 4, 24 and 48 hr. The data are presented as % image area covered by cells at three different timepoints. The cell migration assay produced data with a noticeable trend to reduced migration with higher percents of acryloylated dextran in relation to conjugated dextran. At 4 hr, the mean values among the different gels did not vary significantly using ANOVA. Significance was found between the means for the 20% a-dex-/80% c-dex blend and the 60% a-dex/40% c-dex blend, with a P-value of 0.033. The data at 24 hours were similar to the trend in the 4 hr results; however, there was significance between all the means (P=0.00). At this time, the percentage of cells migrating through the membrane without the gel barrier increased significantly. Significance was found between the 20% a-dex/80% c-dex blend and the 80% a-dex/20% c-dex (P=0.00).

At 48 hr, the same trend of decreasing cell migration with increasing acryloylated dextran was seen. However, statistically, the 40% a-dex/60% c-dex blend and 60% a-dex/40% c-dex blends were quite similar. The significance found between the 20% a-dex/80% c-dex blend and the 80% a-dex/20% c-dex still held (P=0.00). However at 48 hr, the percentage values were lower, this may be attributed in part to the decrease in cell dye after cell divisions. Graphically, cell migration appeared to decrease as the acryloylated dextran increased. Acryloylated dextran does not have cleavable sites to allow cell migration. Expectedly, the number of cells going through the gel should decrease as more of these sites were replaced by MMP-stable cross-links than with MMP-cleavable cross-links. To provide a tissue scaffold with limited cell migration, a mixture with more acryloylated dextran is indicated. If more cell migration is desired, a gel with a higher proportion of conjugated dextran with cleavable sites should be used.

### EXAMPLE 4

### Glycidyl Methacrylate Derived Dextran

In particular, the derivatization of free-hydroxyl groups of polysaccharide materials was achieved following conventional techniques described by van Dijk-Wolthuis, et al [Macromolecules 28: 6317-22, 1995], which was chosen for its calculable control over the degree of substitution (DS) of the end product. The level of derivatization was variable and was dependent on the desired physical properties (e.g., viscosity) for the hydrogel end product. Degree of substitution refers to the molar ratio of cross-linking moiety per saccharide residue. The chemical process of producing glycidyl methacrylate-dextran was initiated through the stepwise addition of 25.0 g of dextran (Sigma D-1662, St. Louis, Mo. Molecular Weight 40,000) to 225 ml of dimethyl sulfoxide (DMSO; Mallinckrodt, Hazelwood, Mo. 4948) under a nitrogen atmosphere. Upon dissolution of the dextran, dimethylaminopyridine (DMAP; 5 g, Sigma, D-5640) was also added under a nitrogen atmosphere. To complete the chemical amalgamation, varying volumes of glycol methacrylate (GMA) (Sigma M-115-7) were added to the mixing flasks. Upon addition of all chemical constituents, the solution was mixed for 48 hours at room temperature. Upon complete dissolution, a calculated 3.4 ml of hydrochloric acid (HCl) was added to stop the reaction and to bring the mixture to neutralization. After stopping the reaction, individual solutions were removed and subsequently dialyzed for two weeks. After such time, the contents were plunged in liquid nitrogen and lyophilized. All samples were freeze-dried for three days before removal. Samples were NMR confirmed.

### EXAMPLE 5

### Cell Adhesion Assay

Hydrogel preparations: In a laminar flow hood, initially, gels were formed in each well of a 96-well cell culture plate. The plates were first pre-coated with dextran monolayer in order to improve the wettability and the affinity of the polystyrene sides of the cell culture wells for the hydrogel. A pre-gel (50 μl) consisting of a mixture of dextran-GMA, peptide, and 0.01% of a water soluble photo initiator, 2-Hydroxy-1-[4-(2-Hydroxyethoxy) phenyl]-2-methyl-1-propanone (Ciba Specialty Chemicals, Basel, Switzerland, Lot#3408H1) in phosphate buffered saline (Gibco, without calcium chloride and magnesium chloride) was added to each well and shook for 30 minutes. The molar concentration of the dextran was kept constant, but the concentration of the peptide was varied. Pre-gels with more than 0% peptides formed gel usually within 10 min due to the cross linking, even without exposing to the UV light. After the cross-linking by peptide, an ultraviolet ray lamp (B-100AP Ultra-Violet Products, Inc., Upland, Calif.) of peak wavelength 370 run was used to initiate photo cross-linking of the remaining non reacted acryloylated dextran. In order to remove unbounded peptides, gels were washed with 200 μl PBS (three times) before the cell adhesion step. Each solution was sterilized by filtration through a 0.2 μm filter.

Cell culture: Bovine endothelial cells (BEC)(CRL GMC 7372B, NIGMS Human Genetic Cell Repository) preferably were used in our studies. Cells were maintained in minimum essential medium (MEM) (Gibco, 10370-021) supplemented with 10% FBS (Gemini-Bioproducts, Woodland, Calif., 100-106), 1% antibiotic-antimycotic (Gibco, 15240-062), and 1% L-glutamine (Gibco, 25030-81) in 5% CO₂. Approximately 300 μl of cell suspension in media (15,000 cells/ml) was added to each well of the culture dish. The cultures plates were then incubated at 37° C., 5% CO₂ for 24 hours.

Cell viability assay: After 24 hours, the medium was removed and the cells were washed with PBS. Then two-color fluorescence cell viability assay (Viability/cytotoxicity kit, Molecular Probes, Inc, Eugene, Oreg.) was used to determine the cytotoxic effect of the polymeric materials on BEC cultures. An aliquot of 200 μl consisting of 10 μl of B and 2 μl A (in 5 mL medium) was added to each well and incubated for 30 minutes. Stained samples were examined at 200× magnification via epifluorescence nicroscopy (Leica DM IRB inverted microscope equipped with an Optronics HBO 100 mercury light source) to visualize both viable (fluorescent filter set) and non-viable (Rhodamine filter set) cells. This assay was used qualitatively and quantitatively to assess how the various groups of materials affected cell viability. For quantitative analysis, images were taken from the bottom-most, in-focus cell to the topmost, in-focus cell every 10-20 clicks on the focusing dial. Image Pro Plus v 5.0 was used to build a reconstructed image by taking the brightest pixel through the entire stack. This was necessary due to the fact that the cells growing on the gel were in focus at different heights. The brightest objects in the reconstructed image were counted and analyzed in terms of their size and shape.

Microscopic visualization: Inverted fluorescence microscope (Leica DM IRB) with a CCD camera (Magnifier) and filter cube containing Rhodamine filter set as an excitation filter, was used for all microscopy experiments to detect the live cell stain. A 10x Leica objective was used for all the experiments. Three random images of fixed dimension per group of materials were captured in the center of the well. The percentage of the image covered by live cells (as indicated by the amount of green fluorescence emitted by the culture) was calculated using image analysis software (Image-Pro® Plus 4.1). The percentage values from the independent experiments were compared between each run, and then combined. The groups of materials were compared between each other using ANOVA (α=0.05).

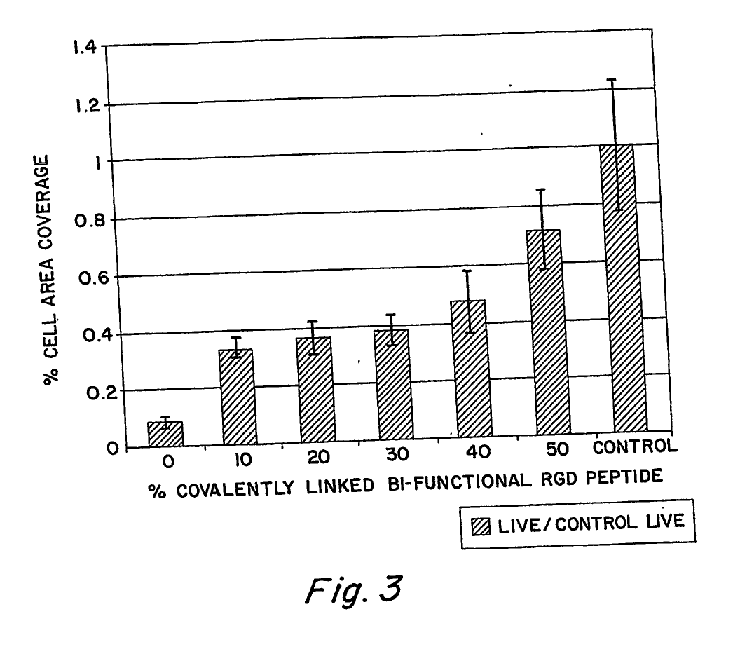

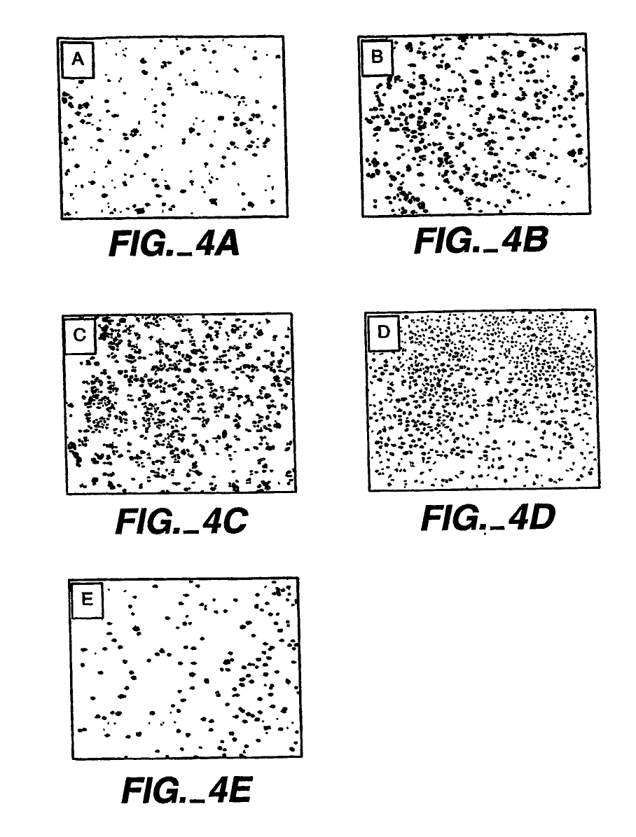

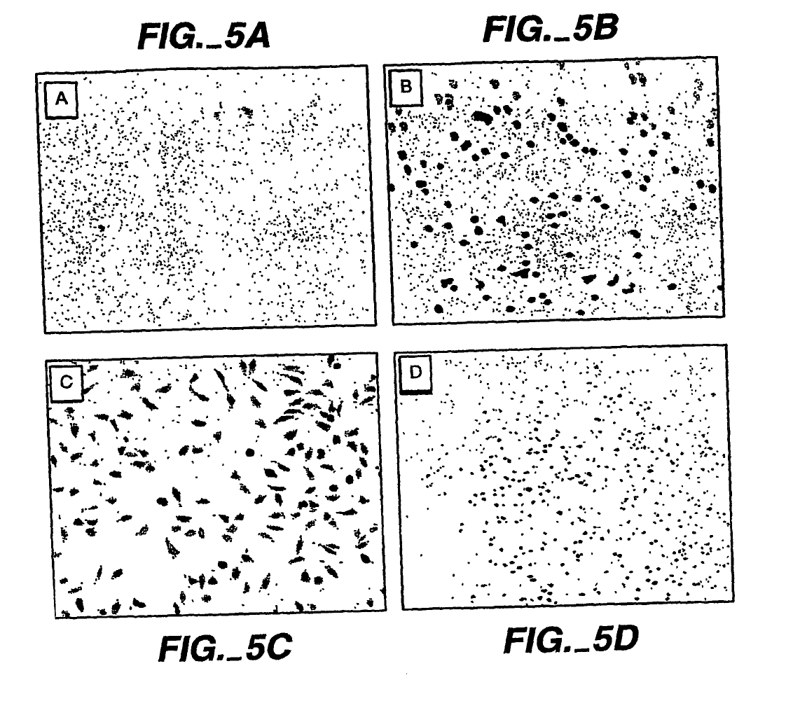

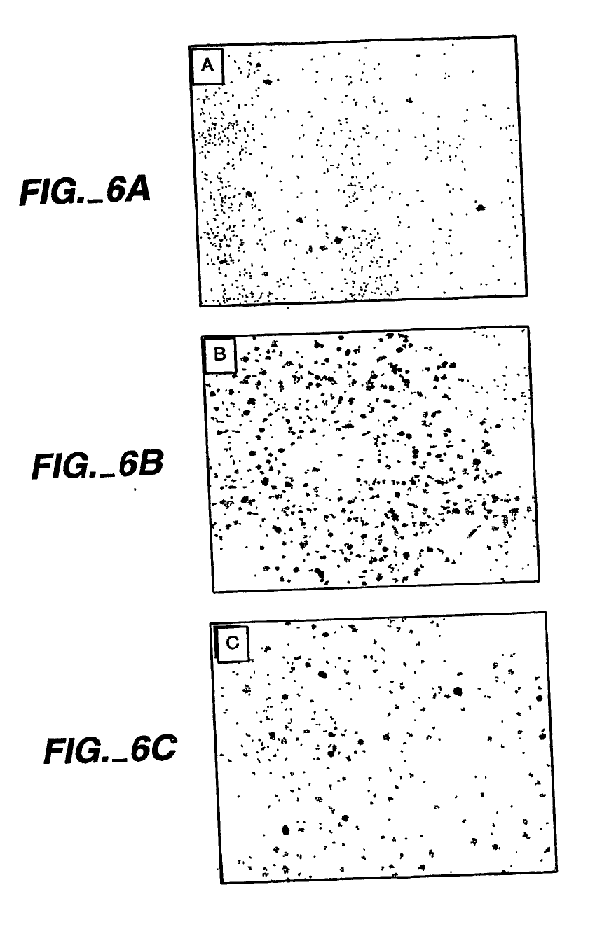

Cell attachment and spreading on the peptide-modified hydrogel surfaces were studied. FIGS. 2a-2c and 3 are typical examples of percentages of cells covering the image area observed at 24 hours. The ratios are given in molar percentages. Bovine Endothelial Cells (BEC) cultured in the Media supplements were visualized 24 h after seeding onto the hydrogels. The morphology of the cells was different. They were round, partially spread, or fully spread depending on the type of coating of the surfaces and on the composition and the conditions of the pre-gel. Qualitatively, cells cultured on the higher percent concentration of the peptide-cross-linked dextran were more spread out when compared with lower percent peptide-cross-linked or non-peptide-cross-linked hydrogels (FIG. 4-6). In summary, cells seeded on the top of the gels containing mono or bi-functional RGD peptides and incubated for 24-72 hours and the extents of adhesion were evaluated. Increases from 10% to 70% area covered by cells based on the percentage of the adhesion peptide within the gel were observed. It was discovered that the gel concentrations with respect to peptide density for cell adhesion can be optimized.

### EXAMPLE 6

### Cell Migration Assay

Hydrogel preparations: Similarly, the cell migration assay was conducted in a laminar flow hood and on a 24-well plate (BD Falcon™ HTS Fluoroblok™ Multiwell Insert System, 8 μm pore size) with a light-opaque membrane of PET. The plates were first pre-wetted with 0.5 ml, of 1% Triton X-100 in PBS in order to improve the affinity of the polystyrene sides of the insert to the hydrogel. The dextran and photo initiator were dissolved in PBS at the 20 wt %. To produce a 1 mm thick gel, 30 μl of the gel mixture is pipetted into each well. Each solution was sterilized by filtration through a 0.2 μm filter.

Cell culture: Bovine Endothelial Cells (BEC) were grown and passaged in minimum essential medium (MEM) (Gibco, 10370-021). Preparation of the cells for use involved exposing cells to a 3 ml solution containing fluorescent probe (CellTracker™ Probes, by Molecular Probes, C-2925, and C-7025). The cells were incubated for 30 minutes and the working solution is replaced with fresh media afterwards. The cells were incubated for an additional 30 minutes. Cells are detached using 0.25% trypsin, 1% EDTA, then pelleted, counted and re-suspended in serum free media at a concentration of 10⁶ cells/mL. The cells were seeded on top of the hydrogels in a volume of 300 μl. The lower wells are filled with 1000 μl of supplemented media via the access ports. The culture plates were then incubated at 37° C., 5% CO₂ for 24 hours.

A preliminary cell migration assay produced data with noticeable trends in reduction of cell migration with higher percentage of acryloylated dextran with respect to covalently degradable peptide cross-linked hydrogels (data not shown). Preliminary experiments showed an increase in cell migration as the concentration of the cleavable peptides was increased.

Conclusion: Attachment and spreading of Bovine Endothelial Cells (BEC) were significantly enhanced on the surface of CRGDSPC (SEQ ID NO: 3) and CRGDSP (SEQ ID NO: 2) modified dextran hydrogels. The concentration and the type of RGD peptides significantly enhanced cell attachment and spreading on the hydrogel surface, demonstrating that surface bioavailability of RGD groups controlled adhesion and spreading. Cells remained viable and proliferated in in vitro culture for at least 94 hours (data not shown). The results suggest that RGD peptide-modified hydrogels have considerable potential for injectable tissue regeneration in vivo. The results from these experiments demonstrated the possibility of controlling cell adhesion/migration using a dextran hydrogel as a barrier.

Also, the following conclusions can be drawn from this investigation: The amount of cell adhesion can be optimized by changing the density of the cross-linking adhesion peptides introduced in a dextran hydrogel. Similarly, the amount of cell migration can be regulated by changing the density of the cross-linking cleavable peptides introduced in dextran hydrogel.

Dextran ratios with higher proportions of acryloylated dextran limited the cell adhesion and consequently migration. Qualitatively, cells cultured on peptide cross-linked hydrogels were relatively more spread when compared with non-peptide cross-linked, and this held for low, moderate, and high-density peptide cross-linked hydrogels. When both adhesion and cleavable peptides are covalently incorporated in the pre-gel, a synergistic effect of cell attachment and migration is expected because the cells will be able to migrate faster through the gel as their secreted proteases attack cleavable cross-links and as the ROD peptide attracts the cells.

Although the invention has been described in detail with reference to the presently preferred embodiments, those of ordinary skill in the art will appreciate that various modifications can be made without departing from the invention. Accordingly, the invention is defined only by the following claims.

1. A biodegradable matrix for inducing cell migration therein, wherein two peptides are covalently linked to the matrix, a first peptide being cleavable by natural proteases and the other comprising a cell-attracting peptide.

2. The matrix of claim 1 wherein the matrix comprises dextran.

3. The matrix of claim 2 wherein the dextran is glycidyl methacrylate dextran.

4. The matrix of claim 2 wherein the dextran has a molecular weight of 40 kDa.

5. The matrix of claim 1 wherein the first peptide comprises at least the sequence CGGLGPAGGLC (SEQ ID NO: 1).

6. The matrix of claim 1 wherein the second peptide comprises at least the sequence RGD.

7. The matrix of claim 6 wherein the RGD sequence further comprises CRGDSP (SEQ ID NO: 2)

8. The matrix of claim 6 wherein the RGD sequence further comprises CRGDSPC (SEQ ID NO: 3)

9. A method of preparing a dextran hydrogel suitable for cellular in-growth, the method comprising
a. providing a dextran;
b. combining the dextran with a cleavable peptide and a peptide capable of attracting cells to produce a conjugated dextran;
c. combining the conjugated dextran with acryloylated dextran;
d. adding to the dextran mixture a polymerization initiator; and
e. activating the initiator to form a hydro gel.

10. The method of claim 9 wherein the provided dextran is dextran 40.

11. The method of claim 9 wherein the cleavable peptide comprises CGGLGPAGGLC (SEQ ID NO: 1).

12. The method of claim 9 wherein the peptide capable of attracting cells comprises at least RGD.

13. The method of claim 12, wherein the RGD peptide further comprises CRGDSP (SEQ ID NO: 2).

14. The method of claim 12, wherein the RGD peptide further comprises CRODSPC (SEQ ID NO: 3).

15. The method of claim 9 wherein the conjugated peptide is in higher proportion than the acryloylated dextran.

16. A method of preparing a hydrogel suitable for promoting cellular in-growth, the method comprising
a. providing dextran;
b. combining the dextran with dimethylsulfoxide (DMSO), dimethylaminopyridine (DMAP) and glycol methacrylate (GMA) to form glycidyl methacrylate dextran;
c. combining the glycidyl methacrylate dextran with acryloylated dextran;
d. combining the dextran mixture with a polymerization initiator and with at least two peptides, a first peptide capable of attracting cells and a second peptide being degradable by cellular proteases, in a dilute electrolyte solution; and
e. applying energy to polymerize the mixture, thus producing a hydrogel.

17. The method of claim 16, wherein step b is performed with the sequential addition of DMSO, DMAP and GMA.

18. The method of 16, wherein step b is followed by mixing at room temperature until the solution is completely dissolved.

19. The method of 16, wherein the mixing step is followed by adding hydrochloric acid to neutralize the solution and stop the reaction.

20. The method of 16, wherein step c is followed by dialyzing the acryloylated dextran.

21. The method of claim 16, wherein the content of the conjugated dextran is greater than the content of acryloylated dextran.

22. An implant comprising a dextran hydrogel, wherein two peptides are covalently linked to the dextran, a first peptide being cleavable by natural proteases and the other comprising a cell-attracting peptide.

23. The implant of claim 22, wherein the dextran has a molecular weight of 40 kDA.

24. The matrix of claim 22, wherein the dextran is glycidyl methacrylate dextran.

25. The matrix of claim 22, wherein the first peptide comprises at least the sequence CGGLGPAGGLC (SEQ ID NO: 1).

26. The matrix of claim 22 wherein the second peptide comprises at least the sequence RGD.

27. The matrix of claim 26 wherein the ROD sequence further comprises CRGDSP (SEQ ID NO: 2).

28. The matrix of claim 26 wherein the RGD sequence further comprises CRGDSPC (SEQ ID NO: 3).

29. A dextran matrix for inducing cell migration therein, wherein a peptide is covalently linked to the matrix, the peptide being cleavable by natural proteases.

30. The matrix of claim 29 wherein the cleavable peptide comprises CGGLGPAGGLC (SEQ ID NO: 1), CGGLGPAGGKG (SEQ ID NO: 4), or a combination thereof.

31. The matrix of claim 29 wherein the dextran is glycidyl methacrylated dextran.

32. The matrix of claim 29 wherein the dextran has a molecular weight of 40 dKa.

33. A dextran matrix for inducing cell migration therein, wherein a peptide is covalently linked to the matrix, the peptide comprising a cell-attracting peptide.

34. The matrix of claim 33 wherein the dextran is glycidyl methacrylated dextran.

35. The matrix of claim 33 wherein the dextran has a molecular weight of 40 dKa.

36. The matrix of claim 33 wherein the peptide is CRGDSP (SEQ ID NO: 2), CRGDSPC (SEQ ID NO: 3), or a combination thereof.
